(19) United States
(12) Patent Application Publication
(10) Pub. No.: US 2003/0170669 A1
(43) Pub. Date: Sep. 11, 2003

(54) Method of nucleic acid recovery

(76) Inventor: Alex M. Garvin, Durmenach (FR)

Correspondence Address: RANKIN, HILL, PORTER & CLARK, LLP, 700 HUNTINGTON BUILDING, 925 EUCLID AVENUE, SUITE 700, CLEVELAND, OH, 44115-1405, US

(21) Appl. No.: 10/257,103

(22) PCT Filed: Apr. 4, 2001
(86) PCT No.: PCT/EP01/03849

(51) Int. Cl. C12Q 1/68; C12N 1/08; C07H 21/04

(52) U.S. Cl. 435/6; 435/270; 536/25.4

(57) ABSTRACT

For reducing side effects of blood transfusion, white (nucleated) blood cells are separated from donated human blood by filtering the blood through filter media selectively adsorbing white blood cells. Such filter media are processed for recovering nucleic acids contained in the white blood cells. The cells retained by the filter media are separated from the filter media e.g. by washing with an aqueous solution. The separated cells are then lysed and the nucleic acids are isolated from the components of the lysed cells. Preferably each portion of filter medium is used for filtering the blood of one individual donor and is processed separately whereby samples of nucleic acids of one individual each are obtained.

The invention concerns a method according to the generic part of the first independent claim. The inventive method serves for recovering nucleic acids from waste material, in particular for recovering human nucleic acids.

Nucleic acids, i.e. DNAs and/or RNAs are the base material for genetic studies. If such studies are to regard human populations, large numbers of nucleic acid samples each representing one human individual are needed. Collecting nucleic acid samples representing a general human population depends on the agreement of each one of a representative plurality of individuals of this population and is therefore very difficult. Similar sampling is carried out on a large scale only in rare cases of crime in which cases relatively small samples of saliva are collected.

The object of the invention is to provide a method for recovering nucleic acids, in particular human nucleic acids, from waste material which method is to be capable to furnish large amounts of nucleic acids for genetic studies or for any other section of science or industry requiring nucleic acids, in particular human nucleic acids.

This object is achieved by the method as defined by the claims.

Based on findings that white blood cells (leukocytes) are the cause of many side effects associated with blood transfusion, blood banks have recently begun removing white blood cells from donated blood. These white blood cells are nucleated cells and therefore constitute a potential source of nucleic acids. The inventive method exploits this source of nucleic acids.

For removing the white blood cells the donated blood is filtered through a filtering medium which selectively adsorbs white blood cells and which allows red blood cells and plasma to pass through. The selectively adsorbing filtering media are obtainable from a number of commercial sources as ready to use, single use filters containing the adsorbing medium. The filtering medium is housed in a rigid, sealed device with two ports, one for whole blood entry and one for removal of leukocyte depleted blood.

For example, Pall Corporation of Port Washington, N.Y., U.S.A., offers a product under the trade name of “Leukotrap WB” that removes more than 99.9% of leucocytes from one unit of whole blood using a polyester based filtering medium. Another supplier of leukocyte depletion filters is the Fenwal Division of Baxter Corporation of Deerfield Ill., U.S.A. (in partnership with Asahi Medical Corporation of Japan) marketing a leukoreduction filter under the trade name of “Sepacell RZ-2000”, which also contains polyester polyfibers selectively adsorbing leukocytes.

For each blood unit taken from one single donor a new filter is used. The red blood cells and plasma passed through the filter are further processed and stored in readiness for blood transfusion or different use and the filter media, instead of being discarded (state of the art) are further processed in order to obtain from them the nucleic acids contained in the white blood cells retained by the filter media through selective adsorption.

The inventive method comprises the following method steps:

(a) filtering blood through a filter medium selectively adsorbing white (nucleated) blood cells;

(b) separating the material retained on filtering at least partly from the filter medium;

(c) isolating nucleic acids from the material separated from the filter medium.

The inventive method is not restricted to obtaining the nucleic acids from human blood donated for transfusion purposes and it is not restricted to obtaining the nucleic acids from filters used and up to now discarded by blood banks, but it is particularly suited to be used in this context. Advantageously each filter having been used for filtering the blood of one donor is processed separately in order to obtain separate nucleic acid samples each representing the genetic material of one human individual.

Step (b) can be carried out by washing the material retained by the filter medium off this filter medium, in particular washing off the white blood cells, or it can be carried out by lysing the cells in-situ, i.e. in the adsorbed state, and washing off the components of the lysed cells from the filter medium.

A preferred way for carrying out step (b) comprises washing the cells off the filter medium using distilled water or an aqueous solution such as phospho-buffered saline or erythrocyte lysis buffer, which is passed through the filter medium preferably in a direction opposite to the filtering direction. The cells can also be separated from the filter medium using elevated temperatures or enzymatic digestion of surface proteins.

As the blood filtering does not constitute a total separation of white and red blood cells the material separated from the filter medium will contain residual red blood cells containing haemoglobin. As haemoglobin interferes with DNA or RNA analysis it is advantageous to remove it from the cell material separated from the filter medium by firstly selectively lysing the red blood cells, by then separating the white blood cells from the components of the lysed red blood cells, e.g. by pelleting through centrifugation, and by then lysing the white blood cells and isolating nucleic acids from the components of the lysed white blood cells.

Selective lysing of the red blood cells can be accomplished using an erythrocyte lysis buffer containing ammonium chloride and potassium hydrogen carbonate. Lysing of the white blood cells can be effected by resuspending the white cell pellet in 3-molar guanidinium hydrochloride. Isolation of DNA and RNA from the cell components is carried out in per se known manner.

For lysing the cells in-situ, i.e. when still adsorbed on the filter medium, a chaotopic agent (e.g. guanidinium hydrochloride) is used or enzymatic digestion or exposure to high temperature. Components of the lysed cells, in particular DNA and RNA are thus released from the cells or filter medium respectively and can be dissolved and removed from the filter medium using an aqueous buffer such as a Tris-EDTA solution. DNA and RNA are purified from this solution in per se known manner.

A single filter through which one donor blood unit has been filtered contains between 2 and 8 billion (2×10¹² to 8×10¹²) nucleated cells each containing DNA and RNA from the same human individual. This means that a large amount of genetic material from a single individual can be gained from each filter.

As a substantial percentage of the general population of societies practising modern medicine take part in blood donation, the genetic material recovered from the blood filters can be looked at as a representative sample of such a society and is therefore of high interest for population based genetic studies.

1. Method for recovering nucleic acids the method being characterized by the steps of: (a) filtering blood through a filter medium selectively adsorbing nucleated cells; (b) separating the material retained on filtering by the filter medium at least partly from the filter medium; and (c) isolating nucleic acids from the material separated from the filter medium.

2. Method according to claim 1, characterized in that said blood is human blood donated by a plurality of individual donors.

3. Method according to claim 2, characterized in that the step of filtering comprises using a separate one of a plurality of filter medium portions for the blood donated by each donor and carrying out the step of separating and the step of isolating for each filter medium portion separately.

4. Method according to one of claims 1 to 3, characterized in that the step of separating comprises liberating adsorbed cells from the filter medium and that the step of isolating comprises lysing the liberated cells.

5. Method according to claim 4, characterized in that the step of separating comprises washing the filter medium with distilled water or with an aqueous solution.

6. Method according to one of claims 4 or 5, characterized in that in the step of isolating, of the liberated cells which comprise white blood cells and residual red blood cells, firstly the red blood cells are selectively lysed, the white blood cells are separated from haemoglobin of the lysed red blood cells and then the white blood cells are lysed.

7. Method according to one of claims 1 to 3 characterized in that the step of separating comprises lysing the cells adsorbed by the filter medium in-situ and dissolving components of the lysed cells.
